(19) United States
(12) Patent Application Publication
(10) Pub. No.: US 2025/0121132 A1
(43) Pub. Date: Apr. 17, 2025

(54) NEEDLE SYSTEM HAVING CLOSED STATE AND OPEN STATE

(71) Applicant: Xaga Surgical AB, Helsingborg (SE)

(72) Inventors: Andreas Forsvall, Helsingborg (SE); Krister Uvnäs, Södra Sandby (SE); Mats Edberg, Eslöv (SE); Rickard Norenstam, Löddeköpinge (SE); Ola Carmonius, Malmö (SE)

(73) Assignee: Xaga Surgical AB, Helsingborg (SE)

(21) Appl. No.: 18/693,285

(22) PCT Filed: Sep. 20, 2022
(86) PCT No.: PCT/EP2022/076102

§ 371 Date: Mar. 19, 2024

(30) Foreign Application Priority Data
Sep. 21, 2021 (EP) EP21198028.9

(51) Int. Cl. A61M 5/158

(52) U.S. Cl. CPC A61M 5/158; A61M 2005/1586; A61M 2005/1587

(57) ABSTRACT

A needle system (10) for injection and aspiration of fluids through a subcutaneous venous port (1) comprises a main body (120) comprising a cavity (121), a conduit (101) with a proximal (211) and distal end (212), a needle section (102) with a proximal (221) and distal end (222), the distal end having an opening (203). The system comprises a rod (103) with a proximal (231) and distal end (232) and a length that is greater than the length of the needle section. The rod comprises an elongated rod shaft portion (106) configured to fit inside the needle section and a rod tip portion (104), at the distal end of the rod. The system comprises a rod control assembly (130). The distal end of the conduit is arranged in the main body and open to the cavity. The proximal end of the needle section is connected to the main body and open to the cavity. The shaft portion is inside the needle section and the proximal end of the rod is attached to the rod control assembly. The rod control assembly is configured to shift the rod in the proximal and distal direction to put the system into a closed state and into an open state. The rod tip portion blocks the distal end opening of the needle section in the closed state, and the rod tip portion is shifted distally relative to the needle section and unblocks the distal end opening of the needle section in the open state, thereby allowing fluid to be injected and/or aspirated through the conduit and the needle section in the open state.

### TECHNICAL FIELD

Embodiments herein relate to a needle system having an open state and a closed state for injection and aspiration through a subcutaneous vein port.

### BACKGROUND

Within the field of medicine, needle systems or needle arrangements for various purposes exist, for example needle arrangements for injection and aspiration. Injection and/or aspiration may take place directly into and out of a vein of a patient. Injection and/or aspiration may also take place via a subcutaneous venous port. In such cases a so-called

Huber needle is often used. A Huber needle can penetrate the membrane of the subcutaneous venous port without cutting out parts of the membrane, which means that the size/mass of the membrane is retained, with the advantage that leakage can be avoided and thereby enabling a long lifetime of the subcutaneous port. Furthermore, avoidance of cutting off parts of the membrane means a reduced risk of unwanted particles entering the bloodstream of a patient.

Nevertheless, Huber needles are associated with drawbacks in that they may carry an undesired number of bacteria when penetrating the skin of the patient and thereby infecting the patient.

### SUMMARY

In view of the above, an object of the present disclosure is to overcome drawbacks related to prior art needle systems for injection and aspiration.

According to a first aspect of the proposed technology, there is provided a needle system for injection and aspiration through a subcutaneous vein port.

According to a second aspect of the proposed technology, there is provided a subcutaneous vein port kit comprising a subcutaneous vein port and a needle system for injection and aspiration through the subcutaneous vein port.

The features of the invention are defined in the independent claims. Preferred alternative configurations of the invention are further defined in the dependent claims.

In the first aspect of the proposed technology, a needle system for injection and aspiration through a subcutaneous vein port is provided. The needle system comprises a main body comprising a cavity, a conduit having proximal end and a distal end, the conduit being arranged along a first longitudinal direction, or first direction, a needle section having proximal end and a distal end, the distal end having a distal end opening, and the needle section being arranged along a second longitudinal direction, or second direction, the second longitudinal direction being different than the first longitudinal direction. The needle system further comprises a rod having a proximal end and a distal end and a length that is greater than the length of the needle section. The rod comprises an elongated rod shaft portion configured to fit inside the needle section and a rod tip portion located at the distal end of the rod. The needle system further comprises a rod control assembly.

The distal end of the conduit is arranged in the main body and open to the cavity. The proximal end of the needle section is connected to the main body and open to the cavity. The rod is arranged such that the rod shaft portion is inside the needle section and the proximal end of the rod is attached to the rod control assembly. The rod control assembly is configured to shift the rod in a proximal direction and a distal direction relative to the main body to put the needle system into a closed state and into an open state. It is understood that the proximal direction is toward the main body and the distal direction is away from the main body. The rod tip portion blocks the distal end opening of the needle section in the closed state and the rod tip portion is shifted distally relative to the needle section and unblocking the distal end opening of the needle section in the open state. Fluid is thereby allowed to be injected and/or aspirated through the conduit and the needle section in the open state.

It is understood that the conduit forms a passage for injection and aspiration to or from the subcutaneous vein port.

The rod control assembly may be configured to provide a proximally directed force on the rod biassing the needle system in the closed state. Such a needle system retains the benefits of prior art, e.g. Huber type, needle systems while providing an advantage in terms of an even further reduced risk of bacteria accompanying the needle during insertion (typically as a consequence of poorly cleansed skin or the presence of crypts in the skin after previous punctures) through the skin of a person into a subcutaneous venous port. In fact, it has been found that some 5% of subcutaneous venous ports today become infected with mainly skin bacteria via skin punctures. Although some infections can be cured by use of antibiotics, in many cases the subcutaneous venous port needs to be urgently surgically removed, with the consequence that an ongoing treatment (often chemotherapy for cancer) must be discontinued. By noting that the rod tip portion blocks the distal end opening of the needle section in the closed state, the needle system of the present disclosure is a means of avoiding or at least minimizing the risk of infections and thereby minimizing the risk of being forced to discontinue treatment of a patient. This advantageous effect is further accentuated if the rod control assembly is configured to provide a proximally directed force on the rod biassing the needle system in the closed state as suggested above.

Moreover, since the needle system is configured with conduit and needle section along different directions, a needle system is thereby provided that can be arranged in a spatially advantageous manner in relation to a subcutaneous venous port. In the needle system, the first longitudinal direction may be perpendicular to the second longitudinal direction. A perpendicular relationship between the conduit and the needle section enables a syringe or other injection/aspiration means connected to the conduit to be arranged along the body of a patient and thereby, e.g., minimizing the risk of injury due to disruption of the needle system when in use. The different longitudinal directions of the conduit and the needle section also enables a favourable arrangement of the rod control assembly. Since the needle system sometimes is attached to a person during several days, it is very practical that the longitudinal directions of the conduit and the needle section are such that a minimal protrusion from the body of the person can be obtained, thereby increasing the comfort, and minimizing the risk of dislocating the needle system from the person.

The rod control assembly may be arranged at least partly within the main body and/or the rod control assembly may be arranged at least partly outside the main body.

The conduit may be a passage integrally formed in the main body. This provides for a cost and time effective manufacturing of the needle system.

The conduit may comprise a second needle section. It is understood the “needle section” as described above is a first needle section. The provision of the first and the second needle sections is advantageous as it provides for possibility of combining a first and a second needle section of different dimensions and attributes. The needle sections may each be straight. It is understood that the first and the second needle sections are arranged, or configured, to allow a sequential passage of a fluid. Worded differently, the first and the second needle sections form a joint fluid passage.

The rod control assembly may be configured to shift the rod in the proximal and distal direction by means of a mechanical system that is configured to convert a rotating motion of a first rod control assembly part around an axis along the second longitudinal direction into a shifting motion proximally and distally along the second longitudinal direction.

An advantageous effect of any such needle system is that an operator of the needle system will find that it is practical and intuitive to use, it is compact and does not take up much space. It is also easy to configure the control assembly such that it is lockable in an open and in a closed position, and it is also easy to provide parts of the control assembly with visual markings that indicate that needle system is open or closed.

The rod control assembly may comprise a rod holder and a cap, said cap forming the first rod control assembly part. The main body may be circular cylindric with a longitudinal axis and configured be arranged with the longitudinal axis along the second direction. The main body may comprise a circumferential surface that comprises a groove having a gradient the direction of the longitudinal axis. The cap may comprise a circular cylindric inside surface with a longitudinal axis, the inside surface comprising at least one protrusion, the cap being configured to be arranged with the longitudinal axis along the second direction and configured to be arranged in relation to the main body such that the inside surface of the cap encloses the circumferential surface of the main body and such that the at least one protrusion fits within the groove. The rod holder may be arranged between the main body and the cap and configured to hold the proximal end of the rod.

Alternatively, the rod control assembly may be configured to shift the rod in the proximal and distal direction by means of a mechanical cam assembly that is configured to convert a rotating motion of a first cam assembly part into a shifting motion proximally and distally along the second longitudinal direction. Such needle system has at least similar advantageous effects as for the needle system suggested above.

It is advantageous that the main body is circular cylindric because it makes it convenient and intuitive to handle. Moreover, as the needle system is often covered by a bandage during use, or plaster, the absence of corners makes the bandage or plaster last longer. Additionally, such needle system is more comfortable for the patient.

The at least one protrusion is advantageous as it contributes to stability of the needle system. Such a protrusion combined with a recess provides a locking effect that prevents the cap from inadvertent rotation.

Arranging the rod holder between the main body and the cap is advantageous as it decreases risk of an unintentional impact on the rod holder, and, thereby, the state of the needle system.

The rod control assembly may comprise a rod holder and a cam, said cam forming the first rod control assembly part. The cam may be rotatably arranged in relation to the main body. The rod holder may be arranged between the main body and the cam and configured to hold the proximal end of the rod.

As described above, the rod control assembly may be configured to provide a proximally directed force on the rod biassing the needle system in the closed state. Worded differently, the rod control assembly may be configured to lock, or bias, the needle system in the closed state. This contributes to prevent unintentional injection or aspiration. The rod control assembly may be configured to lock, or bias, the needle system in the open state. This allows for uninterrupted injection or aspiration. It is understood that the rod control assembly is configured to transition the needle state from the open state to the closed state and vice versa, for example by a manually operated mechanism.

The needle system may further comprise a spring arranged in relation to the rod holder and the main body. The spring may be a coil spring or a leaf spring. The spring may be arranged between the rod holder and the main body. The spring contributes to, or is involved in the process of, transitioning the needle system from one state to another, as described in the detailed description.

The needle section may comprise an outside surface coating for providing a desired friction between the needle section and skin and/or between the needle section and the subcutaneous vein port during insertion into the subcutaneous vein port.

The needle system may provide an additional advantage in that a surface coating that provides a minimal friction also provides an advantageous effect in that it minimizes the adhesion of bacteria. Such an advantage is further appreciated in situations where the needle system is attached to the skin of a person during long periods of time, e.g. several days.

The rod tip portion may be configured with a plurality of facets that create cutting edges. Such needle system has advantageous effects in that cutting edges that facilitate insertion into the subcutaneous vein port in may be combined with an appropriate surface friction between the needle section and the skin to minimize bacteria transport into the subcutaneous vein port.

The needle system may further comprise an elongated tip guard having a proximal end and a distal end, said proximal end being attached to the main body. The tip guard is configured to be set in a guarding state in which the distal end of the tip guard at least partly encloses the rod tip portion.

A needle system and modifications thereof, having the same and additional effects and advantages as those summarized above, will be described in the following detailed description.

Wording the first aspect of the proposed technology differently, a needle system for injection and aspiration through a subcutaneous vein port is proposed that comprises:
a main body,
a single conduit arranged in the main body and having a first conduit portion and a second conduit portion connected, or joined, to the first conduit portion, the first conduit portion being arranged along a first longitudinal direction, or first direction, the second conduit portion being arranged along a second longitudinal direction, or second direction, the second longitudinal direction being different than the first longitudinal direction,
a needle, or a tube, having proximal end, or portion, and a distal end, or portion, the distal end having a distal end opening, and the needle being arranged along the second longitudinal direction,
a rod having a proximal end, or portion, and a distal end, or portion, and a length that is greater than the length of the needle, wherein the rod comprises an elongated rod shaft portion configured to fit inside the needle and a rod tip portion, located at the distal end of the rod,
a rod control assembly,

wherein:
the proximal end of the needle is connected to the main body,
the rod is arranged with the rod shaft portion inside the needle, and the proximal end of the rod is attached to, or operationally connected to, the rod control assembly,
the rod control assembly is configured to shift the rod in a proximal direction and a distal direction relative to the main body to put the needle system into a closed state and into an open state, wherein the rod tip portion blocks the distal end opening of the needle in the closed state, and the rod tip portion is shifted distally relative to the needle and unblocking the distal end opening of the needle in the open state, thereby allowing fluid to be injected and/or aspirated through the conduit and the needle in the open state.

It is understood that the conduit may be tube-like. This means that the conduit has a form of a hollow tunnel. The expression “a single conduit” is understood as consisting of only one in number. In extension, this means that there is one fluid inlet and one fluid outlet provided. Worded differently, the conduit consists of one single channel having two openings. It is further understood that the conduit may be integrally formed in the main body.

The first and the second conduit portions may be straight. This is advantageous as this provides for a smooth, or uniform, flow during injection and aspiration. The rod may extend through the second conduit portion and exit the conduit at the connection between the first conduit portion and the second conduit portion.

The needle may have all the features of the needle section as described herein. It is understood that the needle is tube-like. This means that the needle is hollow. It is understood that the expression “the rod shaft portion configured to fit inside the needle” means that the rod shaft portion at least partially fits inside the needle. Worded differently, the rod shaft portion may be partially enclosed in, or covered by, the needle. Additionally, the needle may have an opening for the rod shaft other than the distal end opening of the needle. As an example, the needle may have a size of 19 G, 20 G, 22 G, 24 G, or 25 G, wherein G is Gauge.

The needle system may further comprise a conduit tube forming at least a part of the first conduit portion. The conduit tube may extend outside if the main body. The conduit tube may comprise any of the features of the second needle section described above. The needle may form a part of the second conduit portion. This means that the needle extends within the main body. It is understood that the needle may extend from the main body.

It is understood that the single conduit and the needle jointly form a passage for injection and aspiration to or from the subcutaneous vein port.

In the open state, the needle and the rod tip portion may cooperate to restrict a flow through the passage at the distal end opening. This contributes to reduce the distance by which the rod must shift to allow injection or aspiration. This provides for an increased stability of the needle system in the open state. For example, in the open state, the rod tip portion and the needle may define a gap between them. The gap defines a circumferential area that is smaller than the cross-sectional area of the passage at the distal end opening. Here, circumferential area is understood as aligned with an outer surface of the needle at the distal end opening. It is understood that the restriction of the flow may be regulated by the rod control assembly.

The first conduit portion may be straight. Similarly, the second conduit portion may be straight. The conduit may be L-shaped. This means that the first conduit portion and the second conduit portion are at a right angle. The provision of the first and the second conduit portions is advantageous as it allows for possibility of combining a first and a second conduit portions having different, or various, dimensions and attributes. Moreover, the L-shaped conduit provides for the needle system to be arranged along the body of a patient and thereby, e.g., minimizing the risk of injury due to disruption of the needle system when in use.

The first conduit portion or the conduit tube may have an interface, such as threads or bayonet mount for establishing a fluid connection with another medical device, for example, a syringe. This is advantageous as this provides for a secure connection to a device for injection and aspiration. Additionally, such connection may provide for exclusivity which means that only devices with suitable corresponding connector may be connected to the first conduit portion. For example, this decreases the risk of mistakes by connecting an erroneous syringe to the first conduit portion, wherein the content of the syringe may harm the patient.

According to the second aspect of the proposed technology, there is provided a subcutaneous vein port kit comprising a subcutaneous vein port and a needle system for injection and aspiration through the subcutaneous vein port. It is understood that the needle system is a needle system according to the first aspect of the proposed technology.

A subcutaneous vein port, also known as an implantable venous port or Port-a-Cath, has a chamber, or portal, comprising a membrane, or a septum, and a tube, or catheter. It is understood that the subcutaneous vein port is configured for being placed completely under the skin of the patient. The membrane may be a rubber or silicone top. The catheter is preferably flexible and configured for being placed into a vein. The catheter may be a silicone, PVC, polyethylene, or polyurethane catheter. It is understood that the needle system for injection and aspiration is configured for being attached to, or cooperate with, the subcutaneous vein port. The expression “attached” is herein understood as inserting distal end of the needle into the chamber through the membrane.

### BRIEF DESCRIPTION OF THE DRAWINGS

FIG. 1a schematically illustrates a needle system, or a subcutaneous vein port kit, in a closed state,

FIG. 1b schematically illustrates the needle system, or the subcutaneous vein port kit, of FIG. 1a in an open state,

FIG. 1c schematically illustrates alternative configurations of a needle system, or a subcutaneous vein port kit,

FIGS. 1d and 1e schematically illustrate a distal end of a needle and a rod of a needle system in a closed and an open state, respectively,

FIG. 1f schematically illustrates a rod tip portion,

FIGS. 2a and 2b schematically illustrate a respective perspective view and a cross-sectional view of a needle system in a closed state,

FIGS. 2c and 2d schematically illustrate a respective perspective view and a cross-sectional view of the needle system illustrated in FIGS. 2a-b in an open state,

FIGS. 3a-c schematically illustrate perspective views of a respective part of the needle system illustrated in FIGS. 2a-d,

FIGS. 3d-f schematically illustrate cross-sectional views of the respective parts of the needle system illustrated in FIGS. 3a-c,

FIGS. 4a and 4b schematically illustrate a respective perspective view and a cross-sectional view of a needle system in a closed state,

FIGS. 4c and 4d schematically illustrate a respective perspective view and a cross-sectional view of the needle system illustrated in FIGS. 2a-b in an open state, and

FIGS. 5a and 5b schematically illustrate a needle system with a tip guard.

### DETAILED DESCRIPTION

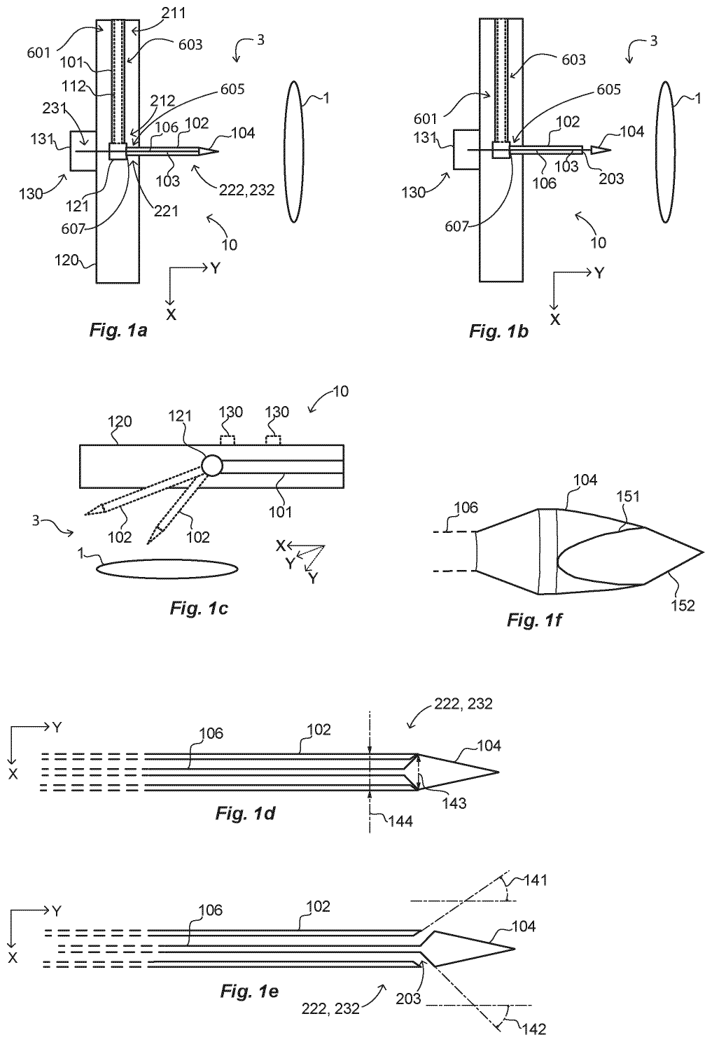

The needle system 10 schematically illustrated in FIG. 1a and FIG. 1b is configured for injection as well as for aspiration of fluids through a subcutaneous venous port 1 as discussed above. The needle system 10 has a main body 120 comprising a cavity 121 and a conduit 101 that has a proximal end 211 and a distal end 212, a needle section 102 that has a proximal end 221 and a distal end 222, the distal end 222 having a distal end opening 203. The needle system 10 further comprises a rod 103 that has a proximal end 231 and a distal end 232 and a length that is greater than the length of the needle section 102. Furthermore, the rod 103 comprises an elongated rod shaft portion 106 configured to fit inside the needle section 102 and a rod tip portion 104, located at the distal end 232 of the rod 103. The needle system 10 further comprises a rod control assembly 130.

The distal end 212 of the conduit 101 is arranged in the main body 120 and open to the cavity 121. The proximal end 221 of the needle section 102 is connected to the main body 120 and open to the cavity 121. The rod 103 is arranged such that the rod shaft portion 106 is inside the needle section 102 and the proximal end 231 of the rod 103 is attached to the rod control assembly 130. The rod control assembly 130 is configured to shift the rod 103 in the proximal and distal direction to put the needle system 10 into a closed state as illustrated in FIG. 1a and into an open state as illustrated in FIG. 1b. The rod tip portion 104 blocks the distal end opening 203 of the needle section 102 in the closed state, and the rod tip portion 104 is shifted distally relative to the needle section 102 and unblocks the distal end opening 203 of the needle section 102 in the open state, thereby allowing fluid to be injected and/or aspirated through the conduit 101 and the needle section 102 in the open state.

The rod tip portion 104 may, as illustrated herein, have a general conical shape with a tip located distally, although other pointed shapes are possible as the skilled person will realize.

As illustrated in FIGS. 1d and 1e, the rod tip portion 104 and the distal end 222 of the needle section 102 may in various embodiments be configured in a manner that has an effect of minimizing friction in relation to the subcutaneous venous port 1 or tissue through which the distal end 222 of the needle section 102 penetrates when in use, in particular during retraction out from the subcutaneous venous port 1. By minimizing friction during retraction, the risk of unintended opening of the needle section 102 is minimized. The rod tip portion 104 may in such embodiments be configured such that its diameter 143 at the distal end opening 203 of the needle section 102 is less than or equal to the outer diameter 144 of the needle section 102 at the distal end opening 203. Further minimizing of friction during retraction may be obtained by configuring the distal end opening 203 with an opening edge angle 141 that is equal or less than an angle 142 of the proximal end of the rod tip portion 104 that abuts the distal end opening 203 when the needle system 10 is in the closed state.

As illustrated in FIG. 1f, the rod tip portion 104 may in various embodiments be configured with a plurality of facets 151 that create cutting edges 152. Such configurations provide an advantageous effect in that less force is needed to insert the needle section 102 through tissue into the subcutaneous venous port 1 and thereby minimizing patient discomfort. Furthermore, such configurations minimize wear on the subcutaneous venous port 1 itself in that little or no material is scraped off the subcutaneous venous port 1 during insertion of the needle section 102. The number of facets 151 may be three, with a corresponding number of cutting edges 152, although the rod tip portion 104 may be configured with other numbers of facets 151. As exemplified in FIG. 1f, the rod tip portion 104 may be configured such that is a combination of a conical shaped tip portion with facets.

With regard to all embodiments herein, it is possible to provide a desired friction between the needle section 102 and skin and/or between the needle section 102 and the subcutaneous venous port 1 during insertion into the subcutaneous venous port 1. Low friction is desirable in order to minimize the force needed to insert the needle section 102, while high friction is desirable in order to minimize the amount of bacteria entering the subcutaneous venous port 1, i.e. bacteria is scraped off at skin level during insertion. The level of friction may be controlled by configuring at least the needle section 102 with a surface coating having appropriate friction characteristics. In any case, a smooth outer surface on the needle section 102 is advantageous in that it minimizes the risk of adhesion of bacteria during insertion of the needle section 102 into the subcutaneous venous port 1. Furthermore, by combining a low friction needle section 102 with a tip portion 104 that provides a relatively higher friction force in relation to tissue during insertion an advantageous effect is obtained in that a minimum amount of bacteria is carried into the subcutaneous venous port 1 during the insertion. For example, an uncoated tip portion 104, with or without facets, provides a relatively higher friction force in relation to tissue during insertion.

The conduit 101 is arranged along a first longitudinal direction X and the needle section 102 is arranged along a second longitudinal direction Y. As illustrated, the second longitudinal direction Y is different than the first longitudinal direction X and, for example, the first longitudinal direction X may be perpendicular to the second longitudinal direction Y as illustrated in FIGS. 1a an 1b. However, as illustrated in FIG. 1c, other angular relations between the first longitudinal direction X and the second longitudinal direction Y are also possible.

The conduit 101 may be a passage integrally formed in the main body 120. In other embodiments, as indicated in FIGS. 1a and 1b by a dashed contour, the conduit 101 may comprise a second needle section 112. Although not illustrated, such a second needle section 112 may extend outside of the main body 120 and, in such embodiments, the conduit 101 may be considered as an interface between the cavity 121 and the second needle section 112.

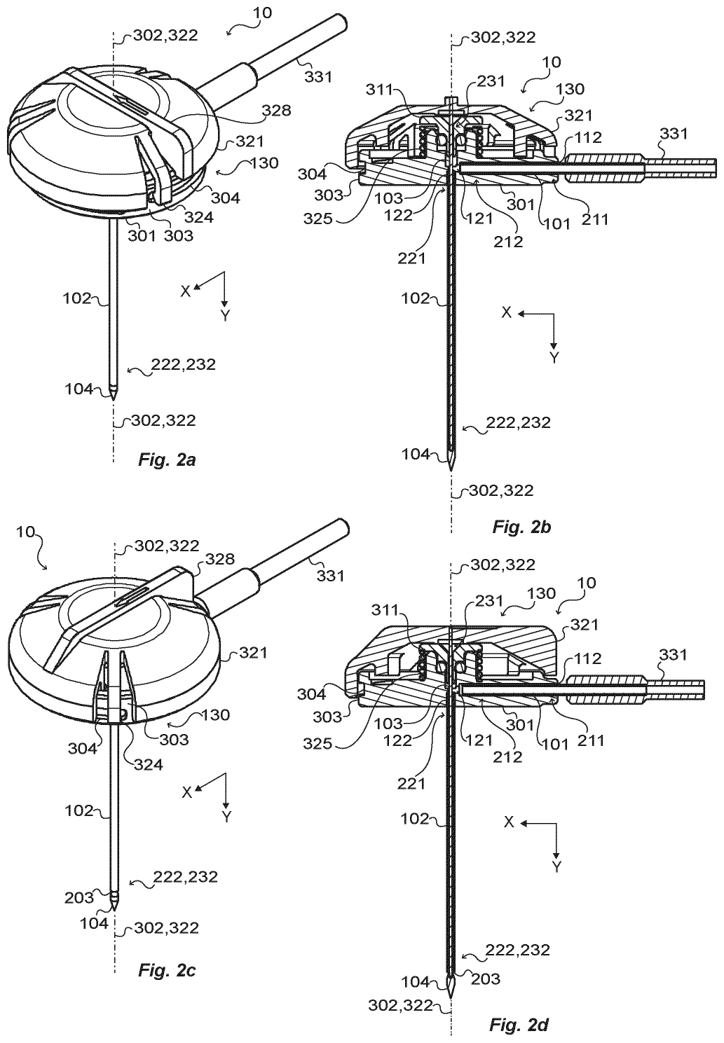

The rod control assembly 130 may be configured to shift the rod 103 in the proximal and distal direction by means of a mechanical system that is configured to convert a rotating motion of a first rod control assembly part 131 around an axis along the second longitudinal direction Y into a shifting motion proximally and distally along the second longitudinal direction Y. Such embodiments are illustrated very schematically in FIG. 1a-c. A more detailed embodiment of such a rotation/translation configuration will be described below in connection with FIGS. 2a-d and 3a-f.

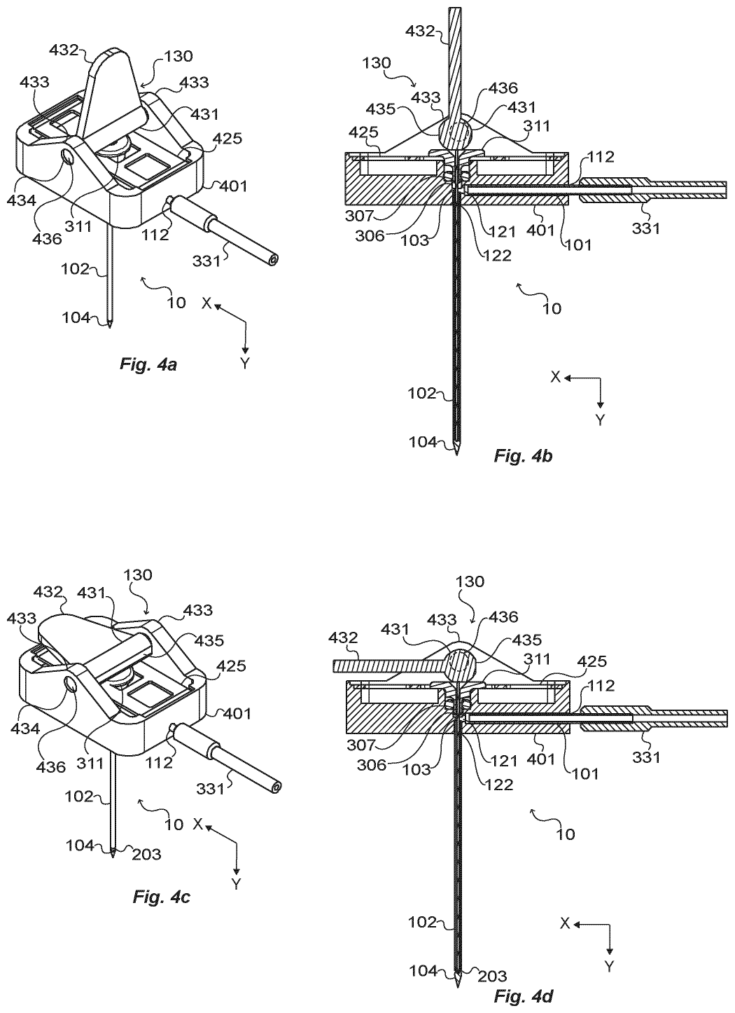

Other embodiments, also very schematically illustrated by FIGS. 1a-c, as will be described in more detail below in connection with FIGS. 4a-d. In such embodiments, the rod control assembly 130 may be configured to shift the rod 103 in the proximal and distal direction by means of a mechanical cam assembly that is configured to convert a rotating motion of a first cam assembly part into a shifting motion proximally and distally along the second longitudinal direction Y.

In any embodiment, the control assembly 130 may be configured to provide a proximally directed force on the rod 103 biassing the needle system 10 in the closed state.

Turning now to FIGS. 2a-d and 3a-f, and with continued reference to FIGS. 1a-c, an embodiment of the needle system 10 will be described in some more detail.

The needle system 10 illustrated in FIGS. 2a-d and 3a-f comprises a main body 301 with a cavity 121, a conduit 101, a needle section 102, a rod 103 and a rod control assembly 130.

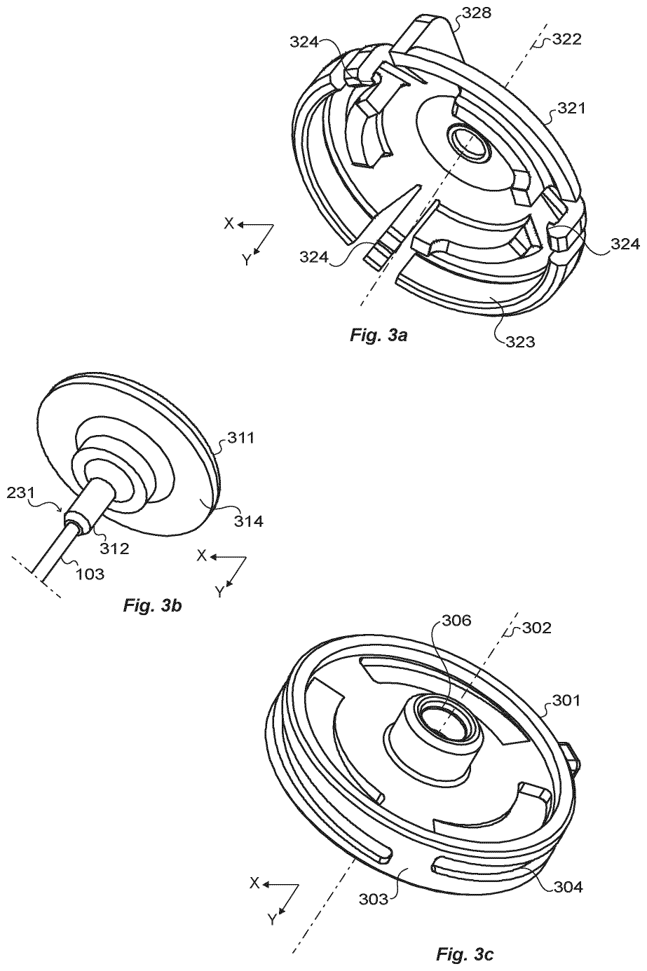

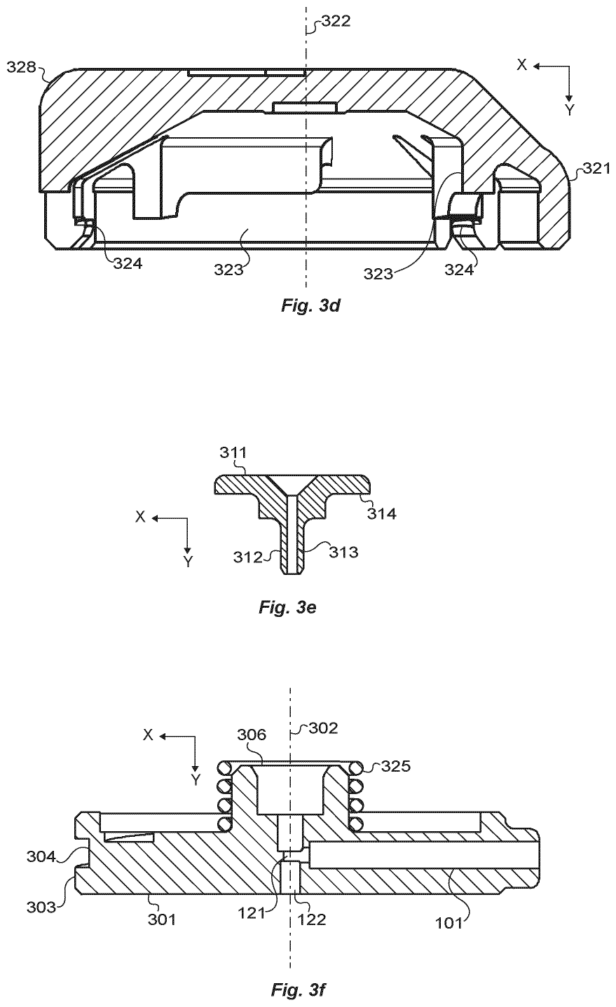

The main body 301 is circular cylindric with a longitudinal axis 302 and it is configured to be arranged with the longitudinal axis 302 along the second direction Y and it comprises a circumferential surface 303 that comprises a groove 304 having a gradient the direction of the longitudinal axis 302. As indicated in FIG. 3c, the groove 304 may encompass nearly the full circumference of the main body 301. However, the extension of the groove 304 along the circumferential surface 303 of the main body 301 may alternatively be shorter and/or divided into separate groove sections.

The conduit 101 along the first direction X opens up to the cavity 121 and a needle connecting recess 122 is configured such that the needle section 102 can be fitted and open up to the cavity 121. A rod holder recess 306 allows room for a rod holder 311 and also for sealing means (not illustrated) for sealing the cavity 121 from the rod holder recess 306. A section of a tube 331 is connected to a second needle section 112 that forms part of the conduit 101. Injection and/or aspiration takes place via the tube 331.

The rod control assembly 130 comprises a rod holder 311 and a cap 321. As such, the rod control assembly 130 is arranged at least partly within the main body 301 and at least partly outside the main body 301, and the cap 321 forms the first rod control assembly part 131 described above. The cap 321 comprises a circular cylindric inside surface 323 with a longitudinal axis 322 and the inside surface 323 of the cap 321 comprises at least one protrusion 324. The cap 321 is configured to be arranged with the longitudinal axis 322 along the second direction Y and configured to be arranged in relation to the main body 301 such that the inside surface 323 of the cap 321 encloses the circumferential surface 303 of the main body 301 and such that the at least one protrusion 324 fits within the groove 304.

Although the cap 321 in the embodiments illustrated in FIGS. 2a-d and 3a-f comprises three protrusions 324, other numbers of protrusions are possible as long as a rotating motion of the cap 321 can ensure a proximal and distal motion of the cap 321 along the second direction Y when the protrusions 324 are forced by the gradient of the groove 304 in the main body 301 to move during rotation of the cap 321 with respect to the longitudinal axis 322. Although not illustrated in the drawings, the cap 321 and the main body 301 may be configured with a further small protrusion/recess combination that can interact and snap into each other at a certain angular relationship between the cap 321 and the main body 301. Such a protrusion/recess combination thereby provides a locking effect that prevents the cap 321 from inadvertent rotation.

In order to facilitate manual rotation of the cap 321, the cap 321 is provided with a grip 328 protruding from the outer surface of the cap 321. Needless to say, the grip 328 may have other configurations as long as the grip 328 enables a user to obtain a secure grip and thereby facilitates rotation of the cap 321.

The rod holder 311, comprising a holding part 312 and a flange part 314, is arranged between the main body 301 and the cap 321 and configured to hold the proximal end 231 of the rod 103 via the bore 313 in the holding part 312. For example, the proximal end 231 of the rod 103 may be fixed by means of glue, which is desirable in embodiments where the rod holder 311 is made of plastic and the rod 103 may be fixed by means of clamping in embodiments where the rod holder 311 is made of metal.

Although the rod holder 311 is exemplified as having a generally circular symmetry with respect to the longitudinal axis 302 of the main body 301 and the longitudinal axis 322 of the cap 321, other non-circular symmetrical shapes of the rod holder 311 are possible. Of course, any such other shape of the rod holder 311 would then correspond to a similarly shaped rod holder recess 306 in the main body 301.

As illustrated in FIGS. 2a-d, the needle section 102 fits in the needle connecting recess 122 in the main body 301 and the rod 103 within the needle section 102 extends through the cavity 121. Furthermore, the rod 103 held in the bore 313 by the holding part 312 of the rod holder 311 that fits, and is movable along the second direction Y, in the rod holder recess 306.

As illustrated in FIGS. 2b, 2d and 3f, in order to provide a proximally directed force on the rod 102 biassing the needle system 10 in the closed state, the control assembly 130 is configured such that a coil spring 325 is arranged in relation to the rod holder 311 and the main body 301, wherein the coil spring 325 is arranged between the rod holder 311 and the main body 301. The spring 325 abuts the rod holder 311 via the flange part 314. When the cap 321 is rotated in a first rotational direction, the protrusions 324 of the cap 321 follow the groove 304 and due to the gradient of the groove 304 the cap 321 is forced distally and the cap 321 thereby provides a force in the distal direction that overcomes the spring force of the spring 325 and, as a consequence, the rod 103 together with its the tip portion 104 move in the distal direction and thereby create the end opening 203 that defines the open state of the needle system 10. When the cap 321 is rotated in a second rotational direction, the protrusions 324 of the cap 321 follow the groove 304 and due to the gradient of the groove 304 and, assisted by the spring force provided by the spring 325, the cap 321 moves in the proximal direction. As a consequence, the rod 103 together with its the tip portion 104 move in the proximal direction and thereby closes the end opening 203 setting the needle system 10 into the closed state.

Turning now to FIGS. 4a-d, and with continued reference to FIGS. 1a-c, another embodiment of the needle system 10 will be described in some more detail.

The needle system 10 illustrated in FIGS. 4a-d comprises a main body 401 with a cavity 121, a conduit 101, a needle section 102, a rod 103, a rod holder recess 306, a needle connecting recess 122 and a rod control assembly 130. The rod control assembly 130 is configured to shift the rod 103 in the proximal and distal direction by means of a mechanical cam assembly that is configured to convert a rotating motion of a first cam assembly part 431 into a shifting motion proximally and distally along the second longitudinal direction Y.

Similar to the embodiments described above, the conduit 101 along the first direction X in the main body 401 opens up to the cavity 121 and the needle connecting recess 122 is configured such that the needle section 102 can be fitted and open up to the cavity 121. The rod holder recess 306 allows room for the rod holder 311 and also for sealing means 307 for sealing the cavity 121 from the rod holder recess 306. A section of a tube 331 is connected to a straight second needle section 112 that forms part of the conduit 101. Injection and/or aspiration takes place via the tube 331.

The rod control assembly 130 illustrated in FIGS. 4a-d comprises a rod holder 311, as described in connection with FIGS. 3b and 3e, and a cam 431. The cam 431 is rotatably arranged in relation to the main body 401 and the rod holder 311 is arranged between the main body 401 and the cam 431 and configured to hold the proximal end 231 of the rod 103. As such, the rod control assembly 130 is arranged at least partly within the main body 401 and at least partly outside the main body 401, and the cam 431 forms the first rod control assembly part 131 discussed above.

Although the rod holder 311 is exemplified as having a generally circular symmetry, other non-circular symmetrical shapes of the rod holder 311 are possible. Of course, any such other shape of the rod holder 311 would then correspond to a similarly shaped rod holder recess 306 in the main body 401.

As for the embodiments described above, the control assembly 130 is configured to provide a proximally directed force on the rod 103 biassing the needle system 10 in the closed state. As illustrated in FIGS. 4a-d, to achieve such a biassing force, a leaf spring 425 is arranged in relation to the rod holder 311 and the main body 401, wherein the leaf spring 425 is arranged between the rod holder 311 and the main body 401.

Such a configuration is obtained by means of the main body 401 having opposing protrusions 433 configured with opposing circular recesses 434 into which a cam axle 436 is arranged. The cam 431 is attached to the cam axle 436. Alternatively, the cam 431 may be integrally formed with the cam axle 436. Moreover, although the cam 431 exemplified herein is in the form of a cylindrical body that has an extension parallel with the cam axle 436 and having an extension that is more or less the same as the extension of the cam axle 436, in other embodiments it is foreseen that the cam may have a relatively smaller extension than the cam axle 436 in the direction that is parallel with the cam axle 436.

Similar to the embodiments described above, in order to provide a proximally directed force on the rod 102 biassing the needle system 10 in the closed state as illustrated in FIGS. 4a-b, the leaf spring 425 abuts the rod holder 311 via the flange part 314.

When the cam axle 436 is rotated, the cam 431 rotates from a first rotational state as illustrated in FIGS. 4a-b to a second rotational state as illustrated in FIGS. 4c-d. In the transition to the second rotational state, the cam 432 increases a force in the distal direction upon the rod holder 311 that overcomes the spring force of the leaf spring 425 and, as a consequence, the rod 103 together with its the tip portion 104 move in the distal direction and thereby create the end opening 203 that defines the open state of the needle system 10.

A reverse rotation of the cam axle 436 and the cam 431 from the second rotational state to the first rotational state decreases the force in the distal direction upon the rod holder 311 and the spring force of the leaf spring 425 forces the rod 103 together with its the tip portion 104 to retract in the proximal direction and thereby closes the end opening 203 returning the needle system 10 to the closed state.

In order to facilitate manual rotation of the cam 431, the cam 431 is provided with a lever 432 protruding from the outer surface of the cam 431. Needless to say, the lever 432 may have other configurations as long as the lever 432 enables a user to obtain a secure grip and thereby facilitates rotation of the cam 431.

Moreover, as exemplified in FIGS. 4a-d, the cam 431 may be configured with flattened peripheral surface areas 435 that provide an effect of locking the cam in the first and second rotational states in order to prevent that an inadvertent pull or push on the lever 432 results in an undesired rotation of the cam 431.

Although the needle system 10 has been illustrated by the embodiments of FIGS. 2a-d and 3a-f where rotational movement of a control assembly is converted into a distal/proximal motion of a rod tip, and by the embodiments of FIGS. 4a-d where a cam assembly provides distal/proximal motion of a rod tip, other embodiments may involve similar functionality. For example, distal/proximal motion of a rod tip may be provided by suitably configured control assembly parts that operate similar to a twist action ball point pen.

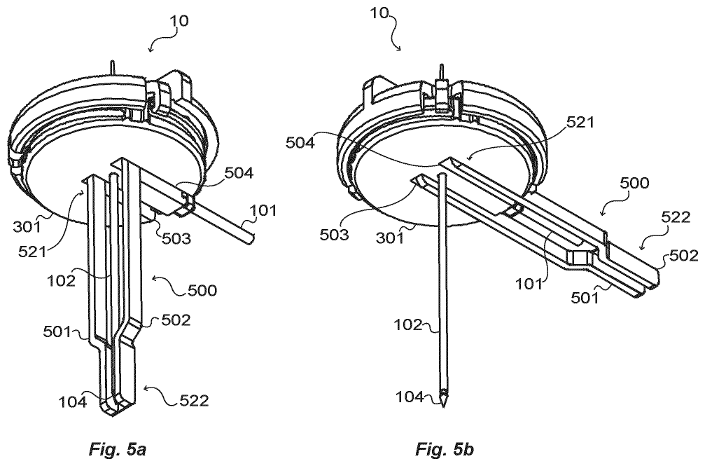

Now, with reference to all embodiments herein, as illustrated in FIGS. 5a and 5b using the embodiment of the needle system 10 of FIGS. 2a-d and 3a-f as an example, the needle system 10 may comprise an elongated tip guard 500 having a proximal end 521 and a distal end 522. The proximal end 521 is attached to the main body 301 and the tip guard 500 is configured to be set in a guarding state in which the distal end 522 of the tip guard 500 at least partly encloses the rod tip portion 104. An advantageous effect of such configuration is that it provides the needle system 10 with a safety function for avoiding injuries to a user of the needle system 10.

As illustrated and exemplified in FIG. 5a, the tip guard 500 may comprise a first elongated section 501 and a second elongated section 502. The elongated sections 501, 502 are in this example blade or leaf shaped and they have spatial extensions and are arranged in relation to each other in the guarding state such that the rod tip portion 104, and here also the needle section 102, is partly enclosed by the elongated sections 501, 502. The proximal end 521 of the tip guard 500, and thereby also the proximal ends of the elongated sections 501, 502, are attached to the main body 301 such that the elongated sections 501, 502 may pivot from the guarding state into a non-guarding state as illustrated in FIG. 5b. The elongated sections 501, 502 are attached to the main body 301 in a respective recess 503, 504 such that when pivoted into the non-guarding state, the elongated sections 501, 502 become flush with the surface of the main body 301. This is advantageous in that it provides a more or less flat surface that facilitates adhering the needle system 10 to the skin of a user/patient on which the needle system 10 is used.

Furthermore, with reference to all embodiments herein, an appropriate choice of material for the various parts of the needle system is a suitable plastic material, although various parts may in some embodiments be made of metal such as exemplified by the rod holder 311 described above.

In another embodiment, the needle system 10, may have the features as described in the embodiments above. As illustrated and exemplified in FIGS. 1a-b, the needle system 10 has a main body 120 of polyurethane, and a single tube-like conduit 601 integrally formed in the main body 120. The needle system is configured for injection and aspiration through a subcutaneous vein port 1. The conduit 601 consists of one single channel and has two openings. The conduit 601 constitutes a straight first conduit portion 603 and a straight second conduit portion 605. The first conduit portion 603 is arranged along a first longitudinal direction X. The second conduit portion 605 is arranged along a second longitudinal direction Y. The second longitudinal direction Y is perpendicular to the first longitudinal direction X. The first conduit portion 603 and a second conduit portion 605 are connected.

As schematically illustrated in FIGS. 1a-b, the needle system 10 has a tube-like 19 Gauge needle 102 of stainless steel with proximal end 221 and a distal end 222. The length of the needle 102 is 25 mm. The distal end 222 has a distal end opening 203. The needle 102 is arranged along the second longitudinal direction Y. The proximal end 221 of the needle 102 is connected to the main body 120.

The needle system 10 has a rod 103 with a proximal end 231 and a distal end 232 and a length of 50 mm which is greater than the length of the needle 102. The rod 103 has an elongated rod shaft portion 106 configured to fit inside the needle 102 and a rod tip portion 104 located at the distal end 232 of the rod 103. The needle 102 has an opening 607 for the rod shaft portion 106 which is opposite the distal end opening 203 of the needle 102.

Additionally, the needle system 10 has a rod control assembly 130. The rod 103 extends through the second conduit portion 605 and exits the conduit 601 at the connection between the first conduit portion 603 and the second conduit portion 605. The first conduit portion 603 and the second conduit portion 605 are straight, at a right angle and form the L-shaped conduit 601.

As illustrated and exemplified in FIGS. 1a-b, the needle 102 forms a part of the second conduit portion 605. The needle 102 extends within the main body 120.

The rod 103 is arranged with the rod shaft portion 106 inside the needle 102. The proximal end 231 of the rod 103 is attached to the rod control assembly 130 by gluing. The rod control assembly 130 is configured to shift the rod 103 in a proximal direction and a distal direction relative to the main body 120 thereby putting the needle system 10 into a closed state and into an open state. The rod tip portion 104 blocks the distal end opening 203 of the needle 102 in the closed state, as illustrated in FIG. 1a. As illustrated in FIG. 1b, the rod tip portion 104 is shifted distally relative to the needle 102 and unblocking the distal end opening 203 of the needle 102 in the open state. Thereby, the fluid is allowed to be injected and/or aspirated through the conduit 601 and the needle 102 in the open state.

The rod control assembly 130 is configured to provide a proximally directed force on the rod 103 biassing the needle system 10 in the closed state, thereby locking the needle system 10 in the closed state, as illustrated in FIG. 1a. Additionally, the rod control assembly 130 is configured to lock the needle system 10 in the open state, as illustrated in FIG. 1b. The rod control assembly 130 is configured to transition the needle system 10 from the open state to the closed state and vice versa by a manually operated mechanism.

As schematically illustrated in an alternative embodiment according to FIGS. 2d and 2b, the needle system 10 has a conduit tube 112 of stainless steel forming a part of the first conduit portion 603 (not shown). The conduit tube 112 extends outside of the main body 301. The conduit tube 112 may comprise any of the features of the second needle section 112 described above.

The first conduit portion 603 has an interface (not shown) in form of bayonet mount for establishing a fluid connection with a syringe (not shown). In an alternative embodiment, the conduit tube 112 has an interface (not shown) in form of bayonet mount for establishing a fluid connection with a syringe (not shown).

The subcutaneous vein port kit 3, as schematically illustrated in FIGS. 1a-1c constitutes a subcutaneous vein port 1 and the needle system 10, as described above.

The subcutaneous vein port 1 has a chamber with a rubber top membrane and a flexible intravenous silicone catheter (not shown). The subcutaneous vein port is configured for being placed completely under the skin of the patient. Additionally, the subcutaneous vein port is configured to be connected to the central venous circulation. The needle system 10 is configured for being attached to the subcutaneous vein port 1 which is provided by inserting the needle into the chamber through the membrane.

1-16. (canceled)

17. A needle system for injection and aspiration through a subcutaneous vein port, the needle system comprising:
a main body having a cavity;
a conduit in the main body and having a proximal end and a distal end, the distal end of the conduit being open to the cavity, the conduit being arranged along a first direction X;
a needle section having a first length between a proximal end in the main body and open to the cavity, and a distal end having a distal end opening, the needle section being arranged along a second direction Y different from the first direction X;
a rod having a second length between a proximal end and a distal end, the second length being greater than the first length, wherein the rod comprises a rod shaft portion configured to fit inside the needle section, and a rod tip portion at the distal end of the rod; and
a rod control assembly to which the proximal end of rod is attached;
wherein the rod control assembly is configured to shift the rod between a proximal position in which the needle system is in a closed state, and a distal position in which the needle system is in an open state, wherein the rod tip portion blocks the distal end opening of the needle section in the closed state, and wherein the rod tip portion is spaced distally from the distal end opening of the needle section to unblock the distal end opening of the needle section in the open state, so as to allow fluid to be injected and/or aspirated through the conduit and the needle section in the open state.

18. The needle system of claim 17, wherein the first direction X is essentially perpendicular to the second direction Y.

19. The needle system of claim 17, wherein the rod control assembly is arranged at least partly within the main body.

20. The needle system of claim 17, wherein the rod control assembly is arranged at least partly outside the main body.

21. The needle system of claim 17, wherein the conduit is a passage integrally formed in the main body.

22. The needle system of claim 17, wherein the conduit comprises a second needle section.

23. The needle system claim 17, wherein the rod control assembly is configured to shift the rod between the proximal position and the distal position by a mechanism configured to convert a rotating motion of a first rod control assembly part around an axis along the second direction Y into a shifting motion proximally and distally along the second direction Y.

24. The needle system of claim 23, wherein:
the rod control assembly comprises a rod holder and a cap, wherein the first rod control assembly part comprises the cap;
the main body is cylindrical with a first longitudinal axis that is arranged along the second direction Y, the main body comprising a circumferential surface defining a groove having a gradient in the direction of the first longitudinal axis;
the cap comprises a cylindrical inside surface around a second longitudinal axis coincident with the first longitudinal axis, the inside surface comprising at least one protrusion, the cap being arranged in relation to the main body such that the inside surface of the cap encloses the circumferential surface of the main body, and such that the at least one protrusion fits within the groove; and
the rod holder is arranged between the main body and the cap and is configured to hold the proximal end of the rod.

25. The needle system of claim 17, wherein the rod control assembly is configured to shift the rod between the proximal position and the distal position by a mechanical cam assembly that is configured to convert a rotating motion of a first cam assembly part into a shifting motion proximally and distally along the second direction Y.

26. The needle system of claim 25, wherein:
the rod control assembly comprises a rod holder and a cam, the cam forming the first cam assembly part;
the cam is rotatably arranged in relation to the main body; and
the rod holder is arranged between the main body and the cam and is configured to hold the proximal end of the rod.

27. The needle system of claim 17, wherein the rod control assembly is configured to provide a proximally directed force on the rod biassing the needle system toward the closed state.

28. The needle system of claim 27, further comprising:
a spring arranged in relation to the rod holder and the main body so as to provide the proximally directed force, wherein the spring is selected from the group consisting of a coil spring and a leaf spring.

29. The needle system of claim 17, wherein the needle section comprises an outside surface coating that provides friction between the needle section and the subcutaneous vein port during insertion into the subcutaneous vein port.

30. The needle system of claim 17, wherein the rod tip portion includes a plurality of facets that are configured as cutting edges.

31. The needle system of claim 17, further comprising a tip guard extending between a proximal end attached to the main body and a distal end, wherein the tip guard is configured to be set in a guarding state in which the distal end of the tip guard at least partly encloses the rod tip portion.
